(19) United States
(12) Patent Application Publication
(10) Pub. No.: US 2024/0209009 A1
(43) Pub. Date: Jun. 27, 2024

(54) PEPTIDES COMPRISING A PHOSPHORYLCHOLINE CONJUGATE AND METHODS OF SYNTHESIZING SAME

(71) Applicant: Tarsier Pharma Ltd., Zichron Yaacov (IL)

(72) Inventors: Chaim EIDELMAN, Tal El (IL); Pieter VAN MEIJEREN, Barneveld (NL); Richard Hendrik BLAAUW, Nijmegen (NL)

(21) Appl. No.: 18/287,886

(22) PCT Filed: Apr. 21, 2022
(86) PCT No.: PCT/IL2022/050413

§ 371 Date: Oct. 22, 2023

Related U.S. Application Data
(60) Provisional application No. 63/177,420, filed on Apr. 21, 2021.

(51) Int. Cl. C07F 9/12; C07K 7/06

(52) U.S. Cl. CPC C07F 9/12; C07K 7/06

(57) ABSTRACT

In one aspect of the invention, there is a compound comprising N-protected tyrosine, modified with phosphorylcholine. Furthermore, use of the compound such as for SPPS, is also provided.

### CROSS REFERENCE TO RELATED APPLICATIONS

This application claims the benefit of priority of U.S. Provisional Patent Application No. 63/177,420, filed on Apr. 21, 2021, the content of which is incorporated herein by reference in its entirety.

### FIELD OF INVENTION

This invention is directed to tyrosine-phosphorylcholine conjugate and uses thereof, such as for synthesis of peptide derivatives.

### BACKGROUND OF THE INVENTION

Ocular inflammation, an inflammation of any part of the eye, is one of the most common ocular diseases. Ocular inflammation refers to a wide range of inflammatory disease of the eye, one of them is uveitis. These diseases are prevalent in all age groups and may be associated with systemic diseases such as Crohn's disease, Behcet disease, Juvenile idiopathic arthritis and others. The inflammation can also be associated with other common eye symptoms such as dry eye and dry macular degeneration. Several drugs have the known side effect of causing uveitis and/or dry eye. The most common treatment for ocular inflammation, is steroids and specifically corticosteroids. However, these treatments have several known and sometimes severe side effects.

Phosphorylcholine (PC) is a small zwitterionic molecule secreted by helminths which permits helminths to survive in the host inducing a situation of immune tolerance as well as on the surface of some bacteria and apoptotic cells. Tuftsin-PhosphorylCholine (TRS) is bi-specific small molecule with immunomodulatory activities. TRS (Thr-Lys-Pro-Arg-Gly-Tyr-PC) is an immunomodulating peptide derivative.

Currently, TRS has been synthesized by post-synthesis modification of Thr-Lys-Pro-Arg-Gly-Tyr, so as to couple the PC moiety to the phenol ring of tyrosine. However, this synthetic approach results in very low yield, thus making the synthesis of TRS ineffective and costly. New simple and efficient methods of synthesizing TRS are highly required.

### SUMMARY OF THE INVENTION

In one aspect of the invention, there is a compound, or a salt thereof, wherein the compound is represented by Formula 1:

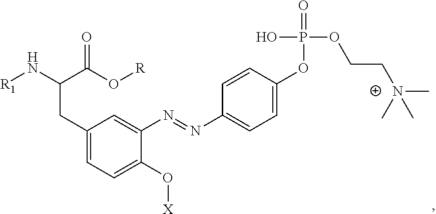

wherein R1 is hydrogen or an amine protecting group; X is hydrogen, a phenol protecting group, or

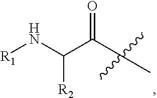

wherein R2 is a side chain of a natural or non-natural alpha amino acid; and R is hydrogen, a carboxyl protecting group, a leaving group, a linker group of a solid phase, or is absent.

In one embodiment, the compound is represented by Formula 2:

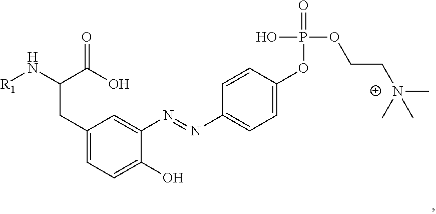

wherein R1 comprises said amine protecting group.

In another aspect, there is a method of synthesizing a compound of interest represented by Formula 3:

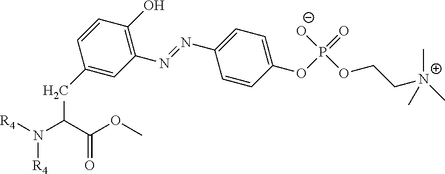

wherein each R4 is independently selected from the group comprising H, an amino acid, a peptide, a polyaminoacid and wherein at least one R4 is not H; and wherein R5 is hydrogen, or a linker group bound to a solid phase; the method comprises providing the compound of the invention linked to a solid phase; and
(i) deprotecting said amine protecting group of said compound;
(ii) coupling a N-protected amino acid moiety to said compound,
(iii) contacting said solid phase with a composition comprising a cyclic amine moiety; thereby obtaining said compound of interest linked to the solid phase.

In one embodiment, the method optionally comprises performing step (iv) of deprotecting said N-protected amino acid moiety prior to performing step (iii).

In one embodiment, the method further comprises subsequently repeating said step (ii) and said step (iv) prior to performing said step (iii).

In one embodiment, the said step (ii) comprises contacting said solid phase with a coupling composition comprising between 1 and 5 molar equivalents of said N-protected amino acid moiety.

In one embodiment, the compound comprises:

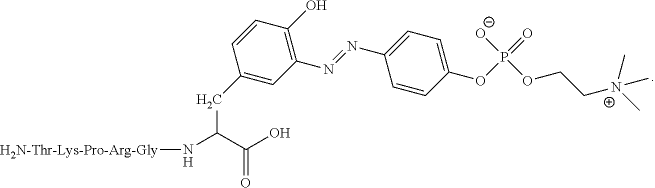

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE FIGURES

FIGS. 1A-1B are graphs depicting an HPLC chromatogram showing impurity profile of TRS synthesized via post-SPPS modification (FIG. 1A); and a corresponding MS spectrum (FIG. 1B) of the major impurity with a retention time of 30.8 min (RRT=1.27). the peaks in the MS spectrum correspond to 422.171 M/Z [M+3H]/3, 632.753 M/Z [M+2H]/2, Mw=1264.576 Da. FIG. 1B presents a proposed structure of the 1.27 impurity (based on MS/MS analysis).

FIGS. 2A-2B are graphs depicting an HPLC chromatogram showing impurity profile of TRS synthesized via a method described herein (FIG. 2A); and a corresponding MS spectrum (FIG. 2B) of the major impurity with a retention time of 17.044 min (RRT=0.7). the peaks in the MS spectrum correspond to 234.0 M/Z [M+2H]/2 and 467.2 M/Z [M+1H]. FIG. 2B presents a proposed structure of the 0.7 impurity (based on MS/MS analysis).

### DETAILED DESCRIPTION OF THE INVENTION

The invention in some embodiments thereof, provides a compound comprising an N-protected tyrosine, modified with PPC via an azo bond. The invention in some embodiments thereof is at least partially based on a surprising finding that the compound of the invention having an unprotected tyrosine side chain can be successfully implemented in the SPPS synthesis of TRS. The inventors surprisingly found that the SPPS synthesis of TRS performed by implementing the compound of the invention (with unprotected phenol group of tyrosine), resulted in a dramatic improvement of the synthesis yield and afforded the desired TRS in high purity.

In one aspect of the invention, there is a compound and/or a salt thereof, wherein the compound is represented by Formula 1:

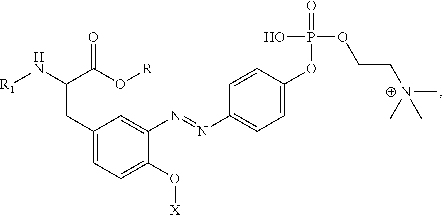

X is hydrogen, a phenol protecting group, or

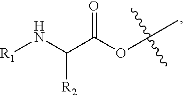

wherein each R1 independently is or comprises hydrogen or an amine protecting group; wherein R2 is a side chain of a natural or non-natural alpha amino acid (protected or unprotected); and R is hydrogen, a carboxyl protecting group, a leaving group, a linker group of a solid phase, or is absent.

In some embodiments, a salt of the compound of the invention comprises a phosphate salt. In some embodiments, the salt of the compound of the invention refers to a phosphate salt of any one the compounds disclosed herein, e.g. deprotonated phosphate group. In some embodiments, the salt of the compound of the invention is represented by Formula I:

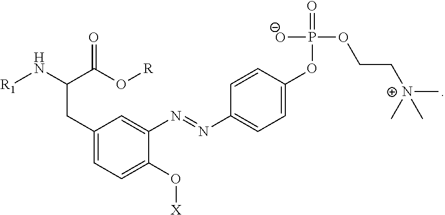

In some embodiments, the salt of the compound of the invention comprises the compound as represented by Formulae 1 or I and a counterion thereof (e.g. a counter cation and/or counter anion).

In some embodiments, the compound of the invention comprises a phosphate salt of the compound represented by Formula 1. In some embodiments, wherein R1 is H, the compound of the invention comprises a phosphate salt and/or an ammonium salt of the compound represented by Formula 1. In some embodiments, the compound of the invention comprises a compound represented by Formula 1 and a counterion. In some embodiments, the counterion comprises a monovalent cation. In some embodiments, the counterion comprises a multivalent cation (e.g. di- and/or tri-valent cation). In some embodiments, the counterion comprises a counter anion (e.g., singly charged counter anion and/or multiply charged counter anion).

In some embodiments, R1 is hydrogen. In some embodiments, R1 is or comprises a protecting group. In some embodiments, R1 is or comprises an amine protecting group. In some embodiments, the amine protecting group is cleavable (or removable) under conditions of solid phases peptide synthesis (SPPS). In some embodiments, the amine protecting group is cleavable (e.g. undergoes deprotection or cleavage resulting in a free unprotected amine) under any of the deprotection conditions applied in SPPS. In some embodiments, the amine protecting group is selected from an acid labile amine protecting group and a base labile amine protecting group.

In some embodiments, the amine protecting group is compatible with SPPS. In some embodiments, the amine protecting group is cleavable under conditions compatible with SPPS. In some embodiments, the amine protecting group is cleavable under conditions appropriate for deprotection of Fmoc or Boc. In some embodiments, the amine protecting group is cleavable under conditions appropriate for Fmoc deprotection. In some embodiments, the amine protecting group is cleavable under conditions appropriate for Boc deprotection. In some embodiments, conditions appropriate for Fmoc or for Boc deprotection refer to conditions applied in solid-phase based synthesis (e.g., SPPS).

In some embodiments, the amine protecting group is cleavable under conditions appropriate for solid-phase Fmoc deprotection. In some embodiments, the amine protecting group is cleavable under conditions appropriate for solid-phase Boc deprotection. Conditions for solid-phase deprotection of Fmoc or Boc are well known in the art. For example, conditions for solid-phase deprotection of Fmoc include inter alia 20% piperidine (or DBU) solution in an organic solvent.

In some embodiments, the amine protecting group comprises any one of 9-fluorenylmethyloxycarbonyl (Fmoc), Alloc, Dde, iv-Dde, benzyl, benzyloxycarbonyl, tert-butyloxycarbonyl (Boc), 2-[biphenylyl-(4)]-propyl-2-oxycarbonyl, dimethyl-3,5dimethoxybenzyloxycarbonyl, 2-(4-Nitrophenylsulfonyl)ethoxycarbonyl, 1,1-Dioxobenzo[b]thiophene-2-ylmethyloxycarbonyl, 2,7-Di-tert-butyl-Fmoc, 2-Fluoro-Fmoc, Nitrobenzenesulfonyl, Benzothiazole-2-sulfonyl, 2,2,2-Trichloroethyloxycarbonyl, Dithiasuccinoyl, p-Nitrobenzyloxycarbonyl.

In some embodiments, the amine protecting group is Boc or Fmoc.

In some embodiments, the compound is as described herein, wherein R1 is devoid of acetyl group. In some embodiments, the compound is as described herein, wherein R1 is devoid of acyl group.

In some embodiments, the carboxyl protecting group comprises a protecting group compatible with SPPS.

In some embodiments, the carboxyl protecting group comprises any one of tert-butyl ester, methyl ester, ethyl ester, benzyl esters, silyl esters (e.g., 2-Trimethylsilylethyl), (2-Phenyl-2-trimethylsiylyl)ethyl, 2-(Trimethylsilyl)isopropyl), allyl ester, 2-Chlorotrityl (2-Cl-Trt), 2,4-Dimethoxybenzyl, 2-Phenylisopropyl, 9-Fluorenylmethyl, Dmab, Carbamoylmethyl, Phenacyl, p-Nitrobenzyl, 4,5-Dimethoxy-2-nitrobenzyl, 1,1-Dimethylallyl. Other carboxyl protecting group are well-known in the art.

In some embodiments, the phenol protecting group comprises a protecting group compatible with SPPS.

In some embodiments, R is or comprises a linker group (also referred to herein as a cleavable linker) attached to a solid phase. In some embodiments, the term “solid phase” and the term “solid support” are used herein interchangeably. As used herein, the term “solid phase” refers to a polymeric resin in a form of particles (usually polymeric beads having a mean diameter ranging from 1 μm to 1 mm). In some embodiments, the solid phase is or comprises a solid phase compatible with the SPPS process (e.g. the solid phase and the linker covalently attaching the compound of the invention, or a propagating peptide chain comprising thereof, are chemically and/or physically stable under conditions applied during the SPPS).

Non-limiting examples of solid supports include but are not limited to PAM, Chlorotrityl, Rink amide, and Wang. Other commercially available resin for solid-phase synthesis (such as peptide synthesis) are well-known in the art.

One skilled artisan will appreciate that a solid phase compatible with the SPPS process further comprise to a cleavable linker. Various cleavable linkers are known in the art, comprising a chemical moiety (e.g. MBHA linker) which allows the compound, or a polypeptide derived therefrom to be cleaved from the solid support.

In some embodiments, the compound, or a polypeptide derived from the compound, is covalently attached to the solid support via the cleavable linker. In some embodiments, the cleavable linker is covalently bound to the solid support and to the compound of the invention, or to a polypeptide derived therefrom. In some embodiments, the cleavable linker is covalently bound to the compound of the invention via the carbonyl group or via the amino group. In some embodiments, the cleavable linker is covalently bound to the compound of the invention via R, wherein R represents a bond. In some embodiments, R represents a covalent bond to a solid phase.

In some embodiments, the compound, or a polypeptide derived from the compound, is covalently attached to the solid support via a cleavable bond. In some embodiments, the cleavable bond is configured to decompose under specific cleavage conditions. The cleavable bond is labile to specific cleavage solutions (usually acidic solution) and is configured to release the compound, or a polypeptide derived therefrom under suitable cleavage conditions such as cleavage solution (usually comprising acidic solutions, such as TFA based solution). In some embodiments, the cleavable bond is labile to any other conditions suitable for cleavage of the cleavable bond, comprising thermal irradiation, UV radiation, exposure to nucleophiles (e.g. hydroxide, alcohol, amine, thiol, hydrazine inter alia under basic conditions).

In some embodiments, the compound of the invention covalently attached to the solid support is represented by Formula 1A:

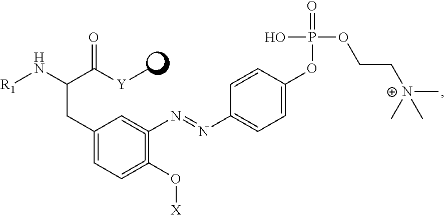

wherein R1 and X are as described herein, Y represents a heteroatom, and [symbol] represents a solid support and/or a linker (i.e., a cleavable linker) attached to the solid support. In some embodiments, Y is selected from O, NH, and S.

In some embodiments, the compound of the invention covalently attached to the solid support is represented by Formula 1A1:

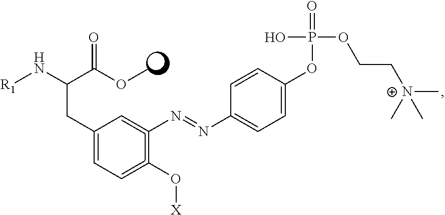

wherein R1 is as described herein. In some embodiments, the compound of the invention is represented by Formula 1A1, wherein R1 represents an amine protecting group. In some embodiments, the compound of the invention is represented by Formula 1A1, wherein R1 is H, Boc, or Fmoc.

In some embodiments, the phenol protecting group comprises any one of triisopropylsilyl ether (TIPS), tert-Butyldimethylsilyl ether (TBDMS), methyl ether, Benzyl ether (Bn), methoxymethyl acetal (MOM), 2-(Trimethylsilyl)ethoxy]methyl acetal, tert-butyl ether, 2-chlorotrityl, trityl, benzyl, benzyloxycarbonyl, Boc.

In some embodiments, the compound of the invention comprises an active ester thereof. Various active esters are known in the art and are generally related to stable derivatives of carboxylates capable of reacting with nucleophiles without any catalyst.

In some embodiments, the active ester of the compound is represented by Formula 1, wherein R is or comprises a leaving group. In some embodiments, the leaving group comprises an active ester obtained by reacting a free carboxy group of the compound of the invention with a coupling reagent. Various coupling reagents are well-known in the art and include inter alia HATU, HOBt, PyBOP, BOP, DIC, DCC, EDAC, etc. In some embodiments, the leaving group comprises any one of halo, hydroxy-succinimide, hydroxybenzotriazole, pentafluorophenol, imidazolecarbonate, O-acylisourea.

In some embodiments, the compound of the invention is further bound to an additional natural or non-natural amino acid. In some embodiments, bound is via a peptide bond. In some embodiments, bound is via the amino group of the compound. In some embodiments, the compound of the invention bound to an amino acid is represented by Formula 1B:

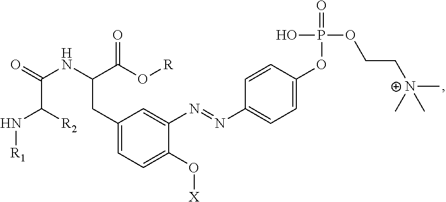

wherein R, R1, and X are as described herein, and wherein R2 is a side chain of a natural or non-natural alpha amino acid (protected or unprotected).

In some embodiments, the compound of the invention is as described herein, wherein R and X are hydrogens. In some embodiments, the compound of the invention is as described herein, wherein R and X are hydrogens and R1 is hydrogen or an amine protecting group.

In some embodiments, the compound of the invention is represented by Formula 2:

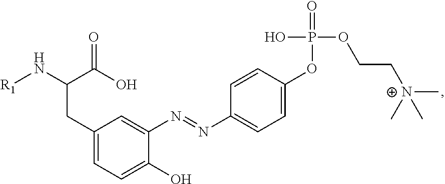

### or by Formula II:

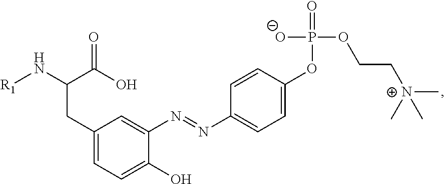

wherein R1 is as described hereinabove.

In some embodiments, R1 is H or the amine protecting group. In some embodiments, R1 is Fmoc or Boc. In some embodiments, the compound of the invention is represented by Formula 2, wherein R1 is Fmoc or Boc. A non-limiting exemplary procedure for the synthesis of one of the compounds disclosed herein is provided in the Examples section.

In some embodiments, there is provided a peptide sequence or a compound of interest comprising a diazotized tyrosine, and is synthesized by the method of the invention; wherein the diazotized tyrosine is represented by Formula 3A:

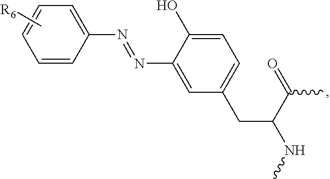

wherein R6 represents one or more substituents, and the wavy bonds represent an attachment point to (i) the peptide sequence of interest; and/or (ii) to any one of R1, N-protecting group, acyl, OR, O⁻, OH and H. In some embodiments, the peptide sequence of interest is represented by Formula 2A:

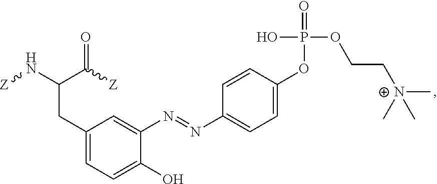

wherein each Z independently is or comprises a peptide, amino acid, NH, OH, or H, and wherein at least one Z is the peptide (e.g. Z bound to the carboxy group).

In some embodiments, the peptide sequence of interest comprises trace amounts of:

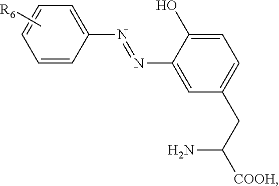

wherein R6 is as described herein. In some embodiments, the peptide sequence of interest is devoid of

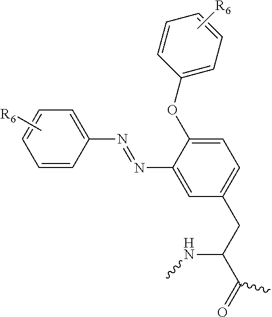

wherein R6 is as described herein, and the wavy bonds represent an attachment point to (i) the peptide sequence of interest; and/or (ii) to any one of R1, N-protecting group, acyl, OR, O⁻, OH and H.

In some embodiments, the peptide sequence of interest is or comprises TRS synthesized by the method of the invention, wherein the TRS comprises at least one impurity of the HPLC impurity profile described in the Examples section, wherein the impurity profile is obtained via an analytical Method A described herein. In some embodiments, the impurity is characterized by a relative retention time (RRT) of 0.7; and/or by MW of 466.4 Da. In some embodiments, the impurity (also referred to herein as the 0.7 impurity) is

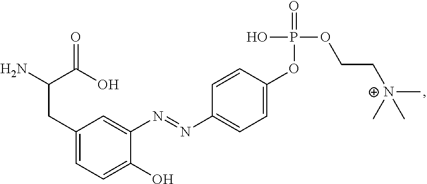 [SMILES: C[N+](C)(C)CCOP(=O)(O)Oc1ccc(/N=N/c2cc(CC(N)C(=O)O)ccc2O)cc1]

including any salt thereof.

In some embodiments, TRS synthesized by the method of the invention is devoid of an impurity (also used herein as 1.27 impurity) characterized by a relative retention time (RRT) of 1.27; and/or by MW of 1263.5 Da, wherein the impurity profile is obtained via an analytical Method A described herein. In some embodiments, TRS synthesized by the method of the invention is devoid of 1.27 impurity:

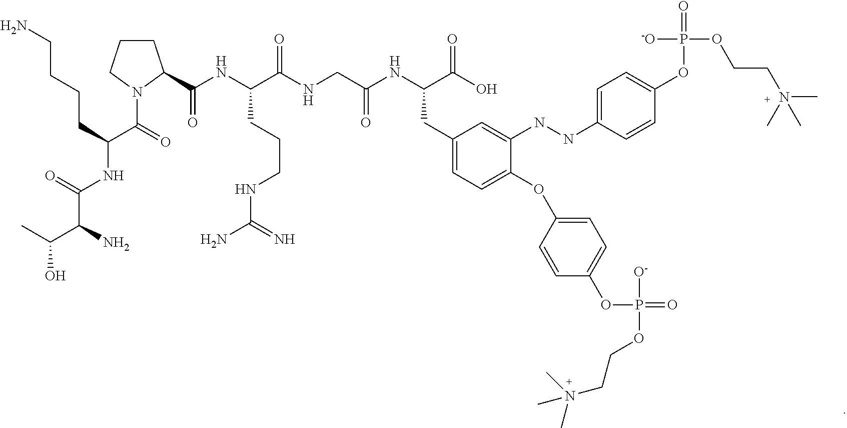

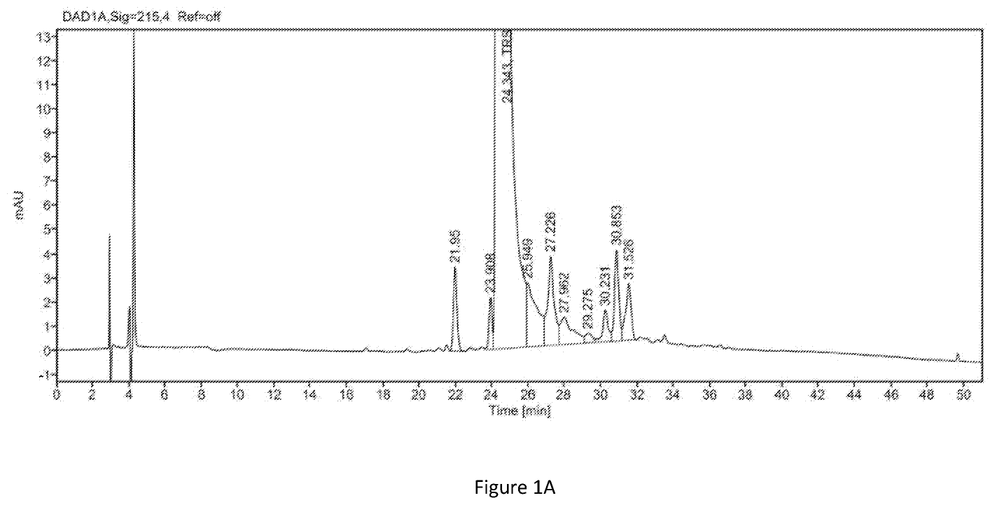

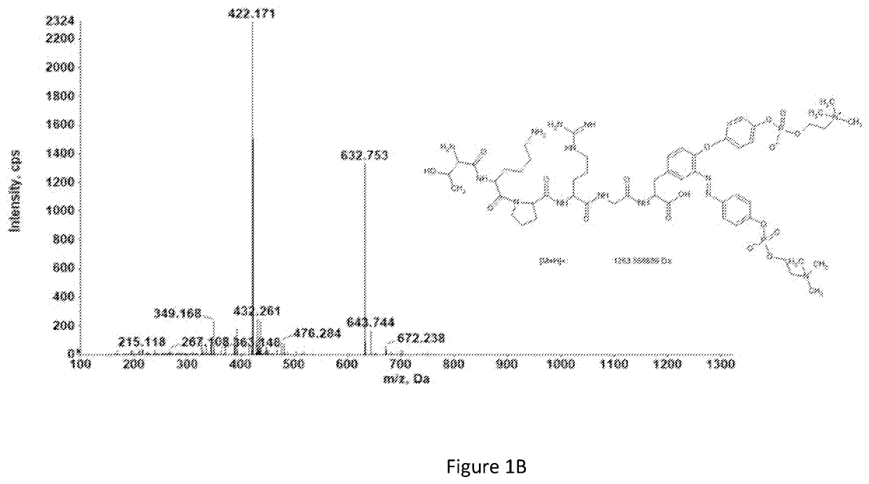

Exemplary HPLC impurity profiles and MS spectra of the impurities are presented in FIGS. 1A-B and 2A-B.

In some embodiments, the peptide sequence of interest synthesized by the method of the invention is characterized by a purity of at least 95%, at least 96%, at least 97%, at least 99%, at least 99.5%, including any range between, as determined by an analytical HPLC. In some embodiments, the peptide sequence of interest synthesized by the method of the invention is substantially pure.

In some embodiments, the terms “peptide sequence”, “peptide sequence of interest” and “peptide of interest” are used herein interchangeably.

As used herein, substantially pure means sufficiently free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

In some embodiments, there is provided a pharmaceutical composition comprising the peptide of interest including any pharmaceutically acceptable salt thereof, wherein the peptide of interest is synthesized by the method of the invention and is a pharmaceutical grade compound. In some embodiments, the pharmaceutical composition comprises the peptide of interest including any pharmaceutically acceptable salt thereof, as a pharmaceutically active agent. In some embodiments, the pharmaceutical composition comprises a therapeutic effective amount of the peptide of interest. In some embodiments, the pharmaceutical composition consists essentially of the peptide of interest, including any pharmaceutically acceptable salt thereof.

In some embodiments, the pharmaceutical composition is for use as a drug (e.g. for treating and/or preventing a disease and/or a medical condition within a subject in need thereof).

In some embodiments, the term “amino acid” encompasses D and/or L-amino acid, optionally comprising one or more protecting groups.

The term “amino acid” as used herein means an organic compound containing both a basic amino group and an acidic carboxyl group. Included within this term are naturally occurring amino acids, protected amino acids (e.g. comprising one or more protecting groups at the carboxyl, at the amine, and/or at the side chain of the amino acid), unusual, non-naturally occurring amino acids, as well as amino acids which are known to occur biologically in free or combined form but usually do not occur in proteins. Included within this term are modified and unusual amino acids, such as those disclosed in, for example, Roberts and Vellaccio (1983) The Peptides. 5: 342-429. Modified, unusual or non-naturally occurring amino acids include, but are not limited to, D-amino acids, hydroxylysine, 4-hydroxyproline, N-Cbz-protected aminovaleric acid (Nva), ornithine (O), aminooctanoic acid (Aoc), 2,4-diaminobutyric acid (Abu), homoarginine, norleucine (Nle), N-methylaminobutyric acid (MeB), 2-naphthylalanine (2Np), aminoheptanoic acid (Ahp), phenylglycine, ß-phenylproline, tert-leucine, 4-aminocyclohexylalanine (Cha), N-methyl-norleucine, 3,4-dehydroproline, N,N-dimethylaminoglycine, N-methylaminoglycine, 4-aminopipetdine-4-carboxylic acid, 6-aminocaproic acid, trans-4-(aminomethyl)-cyclohexanecarboxylic acid, 2-,3-, and 4-(aminomethyl)-benzoic acid, 1-aminocyclopentanecarboxylic acid, 1-aminocyclopropanecarboxylic acid, cyano-propionic acid, 2-benzyl-5-aminopentanoic acid, Norvaline (Nva), 4-O-methyl-threonine (TMe), 5-O-methyl-homoserine (hSM), tert-butyl-alanine (tBu), cyclopentyl-alanine (Cpa), 2-amino-isobutyric acid (Aib), N-methyl-glycine (MeG), N-methyl-alanine (MeA), N-methyl-phenylalanine (MeF), 2-thienyl-alanine (2Th), 3-thienyl-alanine (3Th), O-methyl-tyrosine (YMe), 3-Benzothienyl-alanine (Bzt) and D-alanine (DAI).

As used herein, the terms “peptide”, “polypeptide” and “protein” are used interchangeably, and refer to a polymer of amino acid residues.

The terms “peptide”, “polypeptide” and “protein” as used herein encompass native peptides, peptide derivatives such as beta peptides, peptidomimetics (typically including non-peptide bonds or other synthetic modifications,) and the peptide analogs peptoids and semi-peptoids or any combination thereof. In another embodiment, the terms “peptide”, “polypeptide” and “protein” apply to amino acid polymers in which at least one amino acid residue is an artificial chemical analog of a corresponding naturally occurring amino acid.

The term “derivative” or “chemical derivative” includes any chemical derivative of the polypeptide having one or more residues chemically derivatized by reaction on the side chain or on any functional group within the peptide. Such derivatized molecules include, for example, peptides bearing one or more protecting groups (e.g., side chain protecting group(s) and/or N-terminus protecting groups), and/or peptides in which free amino groups have been derivatized to form amine hydrochlorides, p-toluene sulfonyl groups, carbobenzoxy groups, t-butyloxycarbonyl groups, acetyl groups or formyl groups. Free carboxyl groups may be derivatized to form amides thereof, salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and Dab, Daa, and/or ornithine (O) may be substituted for lysine.

In addition, a peptide derivative can differ from the natural sequence of the peptide of the invention by chemical modifications including, but are not limited to, terminal-NH2 acylation, acetylation, or thioglycolic acid amidation, and by amidation of the terminal and/or side-chain carboxy group, e.g., with ammonia, methylamine, and the like. Peptides can be either linear, cyclic, or branched and the like, having any conformation, which can be achieved using methods known in the art.

### Method

In another aspect, there is method of synthesizing a compound of interest and/or any salt thereof, wherein the compound of interest is represented by Formula 3:

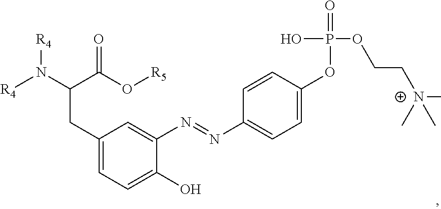

wherein each R4 is independently selected from the group comprising H, an amino acid, a peptide, a polyaminoacid and wherein at least one R4 is not H; and wherein R5 is hydrogen, or a linker group attached toa solid phase; the method comprises: providing the compound of the invention;
(i) deprotecting the amine protecting group of the compound of the invention;
(ii) coupling a N-protected amino acid moiety to the compound of the invention, thereby inducing propagation of the peptide chain attached to the solid phase; and
(iii) contacting the propagating peptide chain with an amine base; thereby obtaining the compound of interest bound to the
   solid phase, and wherein the compound of the invention is or comprises the compound of Formula 1

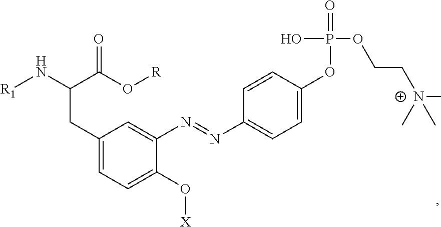

wherein: X is hydrogen, or a phenol protecting group; R1 comprises an amine protecting group; and R represents a linker group attached to a solid phase. In some embodiments, the terms “solid phase” and “solid support” are used herein interchangeably. In some embodiments, the compound of interest comprises a peptide, wherein the sequence of the peptide comprises a diazotized tyrosine described herein (e.g. Tyr-PPC). In some embodiments, the diazotized tyrosine is positioned at the N-terminus, at the C-terminus, and/or within the peptide sequence.

In some embodiments, the method of the invention comprises synthesizing the compound of interest, by providing the compound of the invention attached to a solid phase, and subsequently performing the steps i to iii; wherein the compound of the invention attached to a solid phase is represented by Formula 1A, wherein R1 is or comprises an amine protecting group (e.g. Fmoc), and wherein X is hydrogen and Y is as described herein. In some embodiments, the compound of the invention attached to a solid phase is represented by Formula 1A, wherein R1 is or comprises an amine protecting group (e.g. Fmoc), wherein X is hydrogen; and wherein Y is O. In some embodiments, the steps i-iii are performed in a consecutive order.

In another aspect, the method of the invention comprises providing the compound of the invention attached to a solid phase, and subsequently performing the steps of:
coupling a N-protected amino acid moiety to the N-terminus of the compound of the invention, thereby inducing propagation of the peptide chain attached to the solid phase; and
contacting the propagating peptide chain (and/or the solid phase attached thereto) a sufficient amount of an amine base; thereby obtaining the compound of interest bound to the solid phase , and wherein the compound of the invention is or comprises the compound of Formula 1

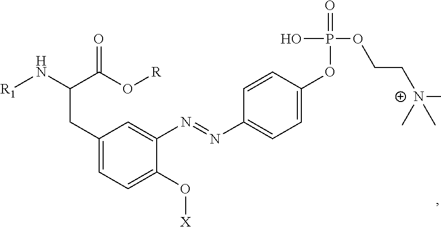

wherein: X is hydrogen, or a phenol protecting group; and R1 is H; and R represents a linker group attached to a solid phase. In some embodiments, X is H.

In another aspect, there is provided a method of synthesizing a peptide sequence of interest represented by Formula A-X-B-NH2, wherein A represents a first amino acid or a first amino acid sequence, B represents a second amino acid or a second amino acid sequence, and X represents:

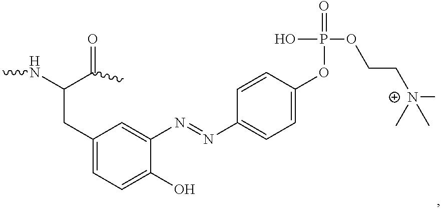

wherein the wavy bonds represent attachment points to the peptide sequences; the method comprising:
providing the first amino acid or a first amino acid sequence bound to a solid phase;
performing a deprotection step to obtain a deprotected first amino acid or a deprotected first amino acid sequence (e.g. comprising a deprotected N-terminal amine);
coupling the deprotected first amino acid or a deprotected first amino acid sequence to the compound of any one of Formulae II or 2, including any salt thereof, wherein R1 comprises an amine protecting group;
deprotecting the amine protecting group to obtain a deprotected N-terminal amine;
coupling the second amino acid or performing a SPPS of the second amino acid sequence, and
subsequently performing the step iii; thereby obtaining the peptide sequence of interest bound to the solid phase.

In some embodiments, the method further comprises performing a cleavage of the peptide sequence, to obtain the peptide sequence of interest.

In some embodiments, each of the steps of the method (e.g. the steps i to iii) is performed by applying a solution of a corresponding reagent. One skilled in the art will appreciate, that solid phase reactions are performed by applying a solution comprising a reagent to the solid support in contact with a propagating chain or with a compound, so as to induce reaction between the reagent and the propagating chain or with the compound. Furthermore, it should be apparent that reactions on solid support require a solvent adopted to provide sufficient swelling to the resin, thereby facilitating reaction or improving reaction yield of each step. Moreover, the solvent has to be compatible with the resin, e.g. the solvent has to be inert to the resin without inducing physical or chemical degradation of the resin. Various solvent can be implemented for solid phase reactions such as DMF, DCM, NMP, etc. Other solvents suitable or compatible with solid phase reactions are known in the art. The exact solvent depends on the chemical composition of the resin, resin swelling, ability of the solvent to dissolve any of the reagents, etc. preferably, dry solvents are used for any one of the steps of the method of invention. Specifically, it is desirable to use dry solvents (water content of less than 1%) for the step ii of the method.

In some embodiments, each of the steps of the method (e.g. the steps i to iii) is performed under conditions sufficient for inducing significant conversion, e.g. reaction yield of at least 50%, at least 60%, at least 70%, at least 80%, at least 90%, at least 95%, at least 97%, at least 99%, including any range between. In some embodiments, each of the steps of the method (e.g. the steps i to iii) is performed under air atmosphere and at a temperature ranging between 10 and 50° C., including any range between. In some embodiments, each of the steps of the method (e.g. the steps i to iii) is performed at a temperature below the boiling point of the solvent.

In some embodiments, each of the steps of the method (e.g. the steps i to iii) is performed once. In some embodiments, any one of the steps of the method (e.g. any one of the steps i to iii) is repeated. In some embodiments, any one of the steps of the method is performed multiple times (e.g. between 2 and 20, between 2 and 4, between 4 and 6, between 6 and 10, between 10 and 20 times including any range between). In some embodiments, any one of the steps of the method is performed once or is performed multiple times until completion of the reaction. One skilled in the art will be able to determine when the reaction is completed. For example, conversion efficiency of the coupling (step ii) can be determined by performing ninhydrin test (also referred to as Kaiser's test). The efficiency of the deprotection step i can be determined by measuring UV-absorbance of the deprotection solution, thus determining the concertation of the cleaved amine protecting group (e.g. for Fmoc deprotection). Furthermore, the duration of any of the steps of the invention can be adjusted to obtain maximum efficiency by monitoring the yield of each step, as described herein.

In some embodiments, step i of the method of invention comprises applying a deprotection solution to the compound or to the propagating chain bound to the solid support for a time period of at least 1 second, at least 1 minute (m), at least 5 m, at least 10 m, at least 20 m, including any range between. In some embodiments, the step i results in amine deprotection or cleavage of the amine protecting group.

In some embodiments, the deprotection solution comprises an appropriate amount of the deprotecting agent (e.g. acid or base) at an amount a sufficient for at least 80%, at least 90%, at least 95%, at least 97%, at least 99%, at least 99.9%, deprotection of the amine protecting group, including any range between. Various deprotection solutions are well known in the art (such as 20% piperidine solution for Fmoc deprotection, or 50% TFA solution for Boc deprotection).

In some embodiments, step ii of the method of invention comprises applying a coupling solution to the compound or to the propagating chain bound to the solid support for a time period of at least at least 1 minute (m), at least 5 m, at least 10 m, at least 20 m, at least 30 m, at least 1 hour, at least 10 h, or more including any range between; wherein the coupling solution comprises a sufficient amount of an amino acid moiety. In some embodiments, step ii of the invention results in a formation of a peptide bond between the amino group and the subsequent amino acid (or results in a coupling of a subsequent amino acid). In some embodiments, step ii of the method of invention comprises coupling an amino acid moiety (e.g. N-protected amino acid moiety) to the amino group of the compound of the invention, thereby inducing chain propagation on the solid phase. In some embodiments, step ii of the method of invention is for propagating the peptide chain. In some embodiments, step ii of the method of invention induces elongation of the growing peptide chain bound to the solid phase.

In some embodiments, the amino acid moiety comprises N-protected amino acid moiety. In some embodiments, the amino acid moiety comprises an active ester of an amino acid. In some embodiments, the amino acid moiety comprises N-protected active ester of an amino acid. Active esters are as described hereinabove. In some embodiments, the amino acid moiety comprises an amino acid.

In some embodiments, the coupling solution comprises a sufficient amount of an N-protected amino acid moiety and optionally a sufficient amount of an organic base (such as DIPEA or collidine). In some embodiments, the coupling solution comprises a sufficient amount of an N-protected amino acid moiety and optionally a sufficient amount of a coupling agent.

In some embodiments, the coupling solution comprises a sufficient amount of an N-protected amino acid and a coupling agent. Coupling agent are well-known in the art, exemplary coupling agents are as described hereinabove. In some embodiments, the coupling solution comprises between 1 and 2 molar equivalents (relative to the amine of the compound or the propagating chain) of the N-protected amino acid and of the coupling agent. In some embodiments, the coupling solution comprises between 1 and 2 molar equivalents of the N-protected amino acid and of the coupling agent and is devoid of a base. In some embodiments, the coupling solution comprises between 1 and 5 molar equivalents, including any range between, of the N-protected amino acid and of the coupling agent, and is devoid of a base.

In some embodiments, the coupling solution comprises between 1 and 5 molar equivalents of the N-protected amino acid moiety, including any range between, and is devoid of the coupling agent and of a base. In some embodiments, the coupling solution comprises between 1 and 5, between 1 and 2, between 2 and 4 molar equivalents of the N-protected active ester of an amino acid (e.g. NHS ester), including any range between, and is devoid of the coupling agent and of a base. In some embodiments, the coupling solution comprises less than 4, less than 3, less than 2, less than 1.6 molar equivalents of a base (e.g. an organic amine base, such as tertiary amine), including any range between.

In some embodiments, the coupling solution comprises between 1 and 6, between 1 and 4, between 1 and 2, between 2 and 4, molar equivalents of the N-protected amino acid moiety. In some embodiments, the coupling solution comprises an N-protected amino acid moiety at an amount a sufficient for at least 80%, at least 90%, at least 95%, at least 97%, at least 99%, at least 99.9%, coupling yield, including any range between. In some embodiments, the coupling solution comprises an N-protected amino acid moiety at a sufficient amount so as to result in a selective coupling to the free amine group of the propagating chain. As used herein, the term coupling selectivity refers to a ratio between coupling to the amino group and coupling to the hydroxy group of the phenol ring of tyrosine.

As represented in Scheme 1, coupling may occur on both the amine group (N-terminus of the propagating chain) and on the OH group of the tyrosine side chain. It is desirable to maximize coupling selectivity, so as to minimize the OH-coupling byproduct.

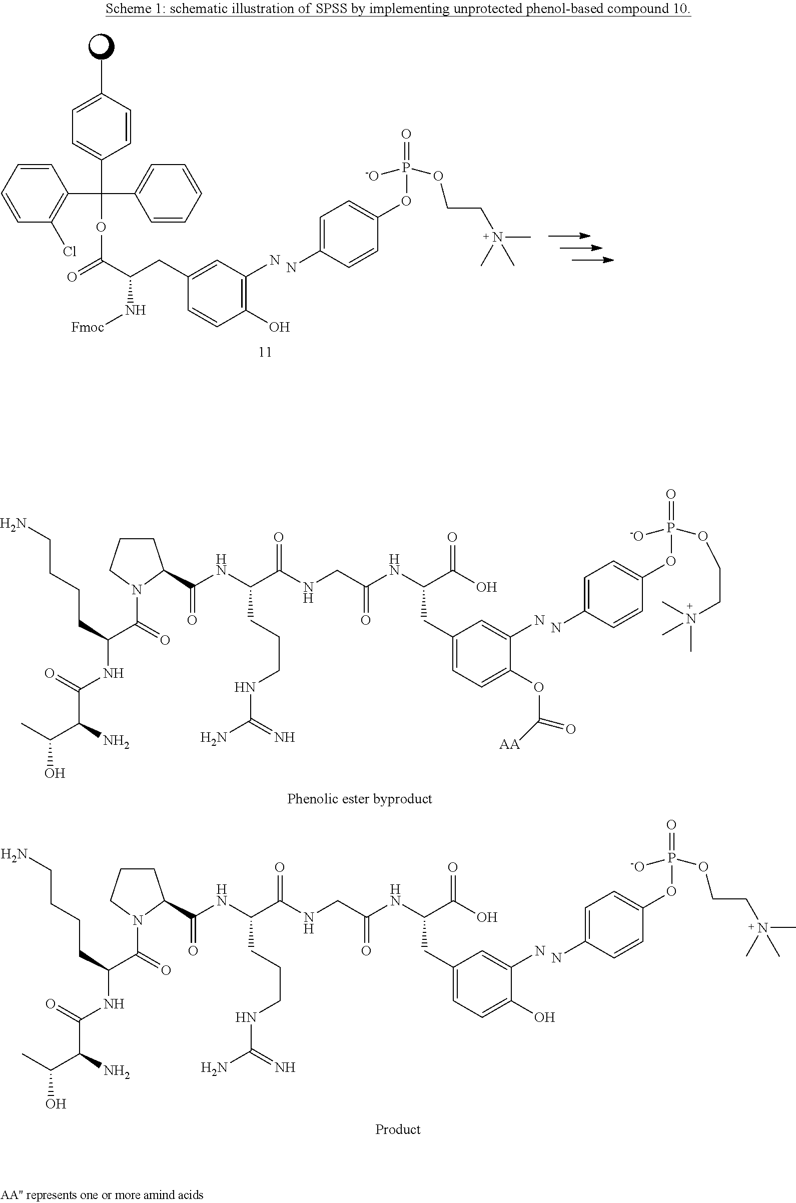

One skill artisan will appreciate, that the exact composition of the coupling solution depends on the amino acid moiety and optionally depends on the specific coupling agent. Furthermore, the exact composition of the coupling solution can be adjusted based on the selectivity and yield of the coupling stage. Non-limiting exemplary coupling solution are as described in the Examples section.

In some embodiments, the steps of the method (e.g. the steps i to iii) are performed in a subsequent order.

In some embodiments, the method of the invention comprises performing a step of loading the compound of the invention on the solid phase, thereby obtaining the compound of the invention attached to a solid phase. Loading is performed according to a well-known procedure.

In some embodiments, the method of the invention comprises performing step iii, thereby removing the byproduct comprising propagating chain coupled to the hydroxy group of tyrosine (e.g. of the phenol ring), as illustrated in Scheme 1 above. As described in the Example section, the inventors performed a novel approach to SPPS of a peptide comprising a diazotized tyrosine (e.g. TRS). As illustrated in Scheme 1, by implementing the SPPS route described herein, the inventors observed a significant byproduct formation due to side reactions occurred on the unprotected phenol ring of the diazotized tyrosine (e.g. compound 13, see Scheme 2 below). Accordingly, in order to obtain the desired product in a sufficient yield, the byproduct has to be cleaved.

To this end, the invention in some embodiments thereof, provides an efficient and simple procedure for the removal of the byproduct (step iii of the invention, as described herein), thus facilitating SPPS synthesis of peptides comprising a diazotized tyrosine, such as TRS.

In some embodiments, step iii comprises contacting the propagating peptide chain with the amine base, or with a solution or composition comprising a sufficient amount thereof. In some embodiments, step iii comprises contacting the propagating peptide chain with a cleavage solution comprising a sufficient amount of the amine base under appropriate conditions, wherein the sufficient amount is so as to induce a substantial (e.g. at least 90%, at least 95%, at least 99%) cleavage or removal of the byproduct. In some embodiments, a sufficient amount comprises at least 1, at least 10, at least 50, at least 100, at least 200, at least 500, between 10 and 500, between 10 and 100, between 50 and 500, between 50 and 100, between 10 and 50, between 1 and 100, between 1 and 10, between 1 and 500, molar equivalents of the amine base, including any range between. In some embodiments, appropriate conditions comprise a contacting time ranging between 1 second and 1 hour, and/or an operable temperature ranging between 5 and 90, or between 15 and 40, or between 15 and 30° C., including any range between.

In some embodiments, the amine base is an Fmoc deprotecting agent. In some embodiments, the amine base is capable of sufficiently deprotecting Fmoc (e.g. resulting in at least 80%, at least 90%, at least 95%, at least 99% Fmoc deprotection on the solid support). In some embodiments, the amine base is selected from a primary amine, a secondary amine, a guanidine-based compound, and an amidine-based compound, including any combination thereof. In some embodiments, the amine base is a linear amine or a cyclic amine.

In some embodiments, the amine base comprises any one of: piperidine, DBU, cyclohexylamine, ethanolamine, pyrrolidine, morpholine, 4-methylpiperidine, tetramethylguanidine, and DBN or any combination thereof.

In some embodiments, the amine base is substantially devoid of a tertiary amine. In some embodiments, the amine base is substantially devoid of DIPEA, TEA or both.

In some embodiments, the method of the invention optionally comprises performing step (iv) of deprotecting the N-protected amino acid moiety prior to performing step (iii). In some embodiments, the step iv comprises applying to the propagating chain bound to the resin a deprotection solution, thereby removing the amine protecting group, wherein deprotection solution is as described herein.

In some embodiments, the method of the invention further comprises subsequently repeating the step (ii) and the step (iv) prior to performing said step (iii). In some embodiments, the step (ii) and the step (iv) are performed and repeated subsequently, thereby propagating the peptide chain. In some embodiments, the step (ii) and the step (iv) are repeated, so as to synthesize a predetermined sequence (e.g. the predefined amino acid sequence). In some embodiments, the step (ii) and the step (iv) are repeated multiple times so as to synthesize a predetermined sequence, wherein multiple times is as described herein.

In some embodiments, the method of the invention further comprises cleaving the synthesized compound (e.g. the peptide) from the solid support.

In some embodiments, the method of the invention is for synthesizing TRS, as represented by Formula 4:

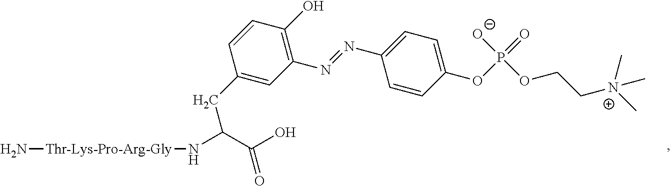

wherein the method comprises the steps i-iii; and further comprises repeating the step (ii) and the step (iv) prior to performing the step (iii), so as to synthesize the peptide sequence represented by Formula 4.

In some embodiments, there is a method of synthesizing TRS, comprising: performing a SPPS, thereby synthesizing a peptide chain on a solid support:

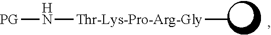

wherein PG is a protecting group (e.g. Fmoc) and wherein the peptide chain further comprises one or more protecting groups on the side chain of the amino acid(s), (ii) subsequently performing a cleavage of the peptide chain (under conditions sufficient for retaining the PG and a protecting group of lysine) thereby obtaining a protected peptide chain:

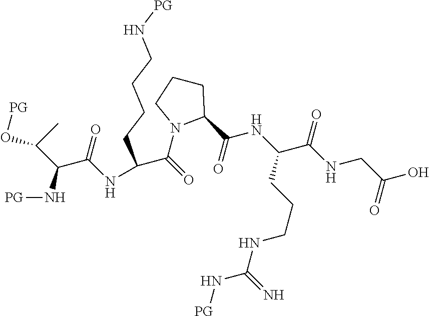

wherein the side chain of lysine, arginine, threonine or both is optionally bound to a protecting group; and (iii) performing a coupling between the protected peptide chain and the compound

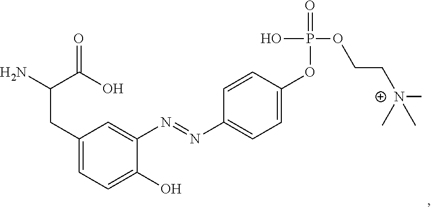 [SMILES: C[N+](C)(C)CCOP(=O)(O)Oc1ccc(/N=N/c2cc(CC(N)C(=O)O)ccc2O)cc1]

thereby obtaining TRS.

In some embodiments, the coupling is performed by mixing the protected peptide chain with a sufficient amount of a coupling solution, thereby obtaining an active ester; and subsequently adding the compound

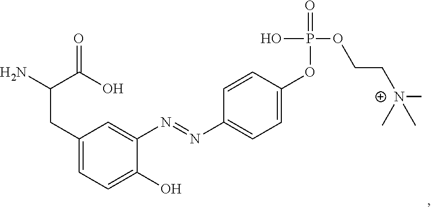 [SMILES: C[N+](C)(C)CCOP(=O)(O)Oc1ccc(/N=N/c2cc(CC(N)C(=O)O)ccc2O)cc1]

to the active ester solution.

In some embodiments, the compound

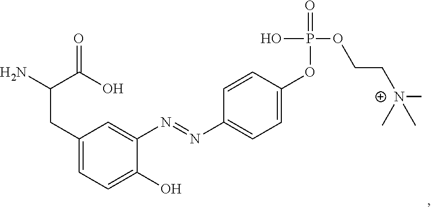 [SMILES: C[N+](C)(C)CCOP(=O)(O)Oc1ccc(/N=N/c2cc(CC(N)C(=O)O)ccc2O)cc1]

is synthesized by providing a compound of Formula 2, wherein R1 is Boc (e.g. a compound described in the Example 1); and deprotecting Boc, as described herein.

In some embodiments, there is a method of synthesizing TRS, comprising: providing a protected peptide chain:

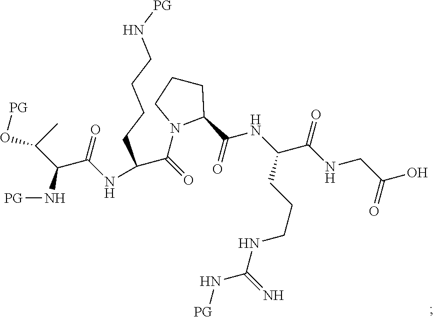

and performing a coupling between the protected peptide chain and the compound

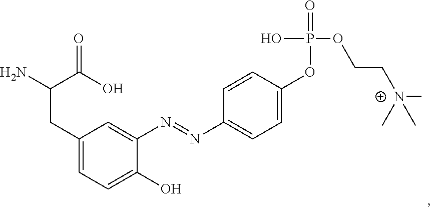 [SMILES: C[N+](C)(C)CCOP(=O)(O)Oc1ccc(/N=N/c2cc(CC(N)C(=O)O)ccc2O)cc1]

thereby obtaining TRS.

In some embodiments, the method comprises providing the diazotized tyrosine bound to the solid support, deprotecting the amine protecting group, thereby obtaining a free amine group; and coupling the free amine group to a subsequent amino acid or polypeptide. In some embodiments, the method further comprises repeating the deprotection step and the coupling step (e.g. multiple times, as described herein), so as to synthesize a predetermined sequence (e.g. the predefined amino acid sequence).

In another aspect, there is method of synthesizing a peptide sequence of interest (including any derivative thereof) comprising a diazotized tyrosine, wherein the diazotized tyrosine comprises a substituent bound to the phenyl ring of the tyrosine via azo bond

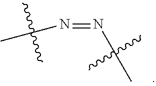 [SMILES: */N=N\*]

In some embodiments, the diazotized tyrosine is or comprises a diazotized tyrosine represented by Formula 3A:

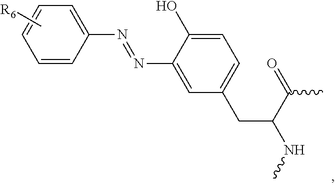

wherein R6 comprises one or more substituents and the wavy bonds represent an attachment point to the peptide sequence of interest; and wherein at least one of the wavy bond represents an attachment point to the peptide sequence. In some embodiments, one of the wavy bonds represents an attachment point to the peptide sequence, and another wavy bond represents an attachment point to (i) any one of R1, N-protecting group, acyl, OR, O⁻, OH and H, (ii) to the peptide sequence of interest.

In some embodiments, the method comprises coupling compound:

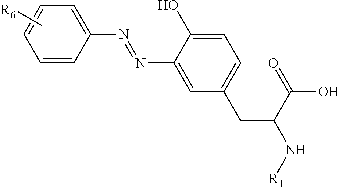

to a propagating peptide chain on a solid support; and performing the step iii, thereby obtaining the peptide sequence of interest bound to the solid support; wherein R1 comprises an amine protecting group, and R6 is as described herein.

In some embodiments, the method further comprises coupling a subsequent N-protected amino acid to an N-terminus, and deprotecting the amine protecting group to obtain a deprotected N-terminal amine; wherein the coupling step and the deprotecting step are performed prior to performing the step iii. In some embodiments, the method comprises repeating the coupling step and the deprotecting step, thereby obtaining the peptide sequence of interest bound to the solid support. In some embodiments, the method further comprises performing a cleavage of the peptide sequence, to obtain the peptide sequence of interest.

In some embodiments, the peptide sequence of interest is represented by Formula A-X—B—NH2, wherein A represents a first amino acid sequence, B represents a second amino acid sequence, and X represents the diazotized tyrosine; the method comprising:
providing the first amino acid sequence bound to a solid phase;
performing a deprotection step to obtain a deprotected first amino acid sequence (e.g. comprising a deprotected N-terminal amine);
coupling the deprotected first amino acid sequence to the compound

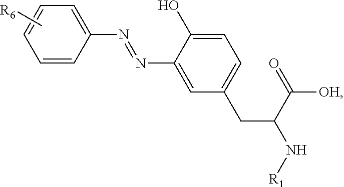

wherein R1 comprises an amine protecting group, and R6 is as described herein;
deprotecting the amine protecting group to obtain a deprotected N-terminal amine;
performing a SPPS of the second amino acid sequence, thereby propagating the peptide chain and
subsequently performing the step iii; thereby obtaining the peptide sequence of interest bound to the solid phase.

In some embodiments, the method further comprises performing a cleavage of the peptide sequence, to obtain the peptide sequence of interest.

In some embodiments, R6 represents a substituent comprising phosphorylcholine, (C₀-C₆)alkyl-aryl, (C₀-C₆)alkyl-heteroaryl, (C₀-C₆)alkyl-(C₃-C₈) cycloalkyl, optionally substituted C₃-C₈ heterocyclyl, halogen, —NO₂, —CN, —OH, —CONH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR₂, C₁-C₆ alkyl-SR, -CONH(C₁-C₆ alkyl), —CON(C₁-C₆ alkyl)₂, —CO₂H, —CO₂R, —OCOR, —OCOR, —OC(═O)OR, —OC(═O)NR, —OC(═S)OR, —OC(═S)NR, a polyaminoacid, a peptide or a combination thereof. Additional substituents are well-known in the art.

In some embodiments, the method comprises performing a SPPS, thereby synthesizing a peptide chain; and coupling the diazotized tyrosine to the peptide chain (e.g. N-terminus of a propagating peptide chain). In some embodiments, the diazotized tyrosine is represented by Formula 3B:

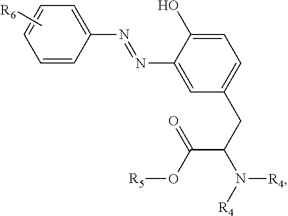

wherein R6 and R4 are as described herein, and R5 is

H or an active ester or absent. In some embodiments, at least one R4 is or comprises an amine protecting group (e.g. Fmoc).

In some embodiments, the method comprises (i) performing a SPPS, thereby synthesizing a peptide chain, (ii) subsequently performing a cleavage of the peptide chain (under conditions sufficient for retaining the protecting group on either N-terminus or C-terminus, and/or on a side chain) thereby obtaining the peptide chain comprising a deprotected N-terminus or a deprotected C-terminus; and (iii) performing a coupling between the deprotected N-terminus or the deprotected C-terminus and the diazotized tyrosine as described herein (e.g. diazotized tyrosine of Formula 3B), wherein step (iii) is performed in the solution (e.g. by utilizing an organic solvent compatible with the reactants).

In some embodiments, the diazotized tyrosine bound to the solid support is represented by Formula 3C:

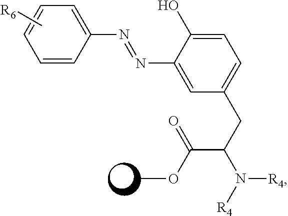

wherein R6 is as described herein, and at least one R4 comprises an amine protecting group.

In some embodiments, the method of the invention further comprises removing the byproduct, by performing the step iii, as described herein, thereby obtaining the predetermined sequence being substantially devoid of the byproduct.

In some embodiments, the method of the invention results in the formation of less than 10, less than 8, less than 5, less than 1, less than 0.5, less than 0.1mol % of the byproduct, including any range between. In some embodiments, the amount of the byproduct relates to molar percentage of the product relative to the total amount of peptide sequences (e.g. bound to the solid support, or after cleavage).

### Definitions

As used herein, the term “alkyl” describes an aliphatic hydrocarbon including straight chain and branched chain groups. Preferably, the alkyl group has 21 to 100 carbon atoms, and more preferably 21-50 carbon atoms. Whenever a numerical range e.g., “21-100”, is stated herein, it implies that the group, in this case the alkyl group, may contain 21 carbon atom, 22 carbon atoms, 23 carbon atoms, etc., up to and including 100 carbon atoms. In the context of the present invention, a “long alkyl” is an alkyl having at least 20 carbon atoms in its main chain (the longest path of continuous covalently attached atoms). A short alkyl therefore has 20 or less main-chain carbons. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine, or iodine.

The term “haloalkyl” describes an alkyl group as defined herein, further substituted by one or more halide(s).

The term “haloalkoxy” describes an alkoxy group as defined herein, further substituted by one or more halide(s).

The term “hydroxyl” or “hydroxy” describes a —OH group.

The term “mercapto” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amino” describes a —NR′R″ group, with R′ and R″ as described herein.

The term “heterocyclyl” describes a monocyclic or fused ring group having in the ring(s) one or more atoms such as nitrogen, oxygen, and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” or “carboxylate” describes a —C(O)OR′ group, where R′ is hydrogen, alkyl, cycloalkyl, alkenyl, aryl, heteroaryl (bonded through a ring carbon) or heterocyclyl (bonded through a ring carbon) as defined herein.

The term “carbonyl” describes a —C(O)R′ group, where R′ is as defined hereinabove.

The above-terms also encompass thio-derivatives thereof (thiocarboxy and thiocarbonyl).

The term “thiocarbonyl” describes a —C(S)R′ group, where R′ is as defined hereinabove.

A “thiocarboxy” group describes a —C(S)OR′ group, where R′is as defined herein.

A “sulfinyl” group describes an —S(O)R′ group, where R′is as defined herein.

A “sulfonyl” or “sulfonate” group describes an —S(O)2R′ group, where R′ is as defined herein.

A “carbamyl” or “carbamate” group describes an —OC(O)NR′R″ group, where R′ is as defined herein and R″ is as defined for R′.

A “nitro” group refers to a —NO2 group.

The term “amide” as used herein encompasses C-amide and N-amide.

The term “C-amide” describes a —C(O)NR′R″ end group or a —C(O)NR′-linking group, as these phrases are defined hereinabove, where R′ and R″ are as defined herein.

The term “N-amide” describes a —NR″C(O)R′ end group or a —NR′C(O)— linking group, as these phrases are defined hereinabove, where R′ and R″ are as defined herein.

The term “carboxylic acid derivative” as used herein encompasses carboxy, amide, carbonyl, anhydride, carbonate ester, and carbamate.

A “cyano” or “nitrile” group refers to a —CN group.

The term “azo” or “diazo” describes an —N═NR′ end group or an —N═N— linking group, as these phrases are defined hereinabove, with R′ as defined hereinabove.

The term “guanidine” describes a —R′NC(N)NR″R′″ end group or a —R′NC(N) NR″— linking group, as these phrases are defined hereinabove, where R′, R″ and R′″ are as defined herein.

As used herein, the term “azide” refers to a —N3 group.

The term “sulfonamide” refers to a —S(O)2NR′R″ group, with R′ and R″ as defined herein.

The term “phosphonyl” or “phosphonate” describes an —OP(O)—(OR′)2 group, with R′ as defined hereinabove.

The term “phosphinyl” describes a —PR′R″ group, with R′ and R″ as defined hereinabove.

The term “alkylaryl” describes an alkyl, as defined herein, which substituted by an aryl, as described herein. An exemplary alkylaryl is benzyl.

The term “heteroaryl” describes a monocyclic (e.g. C5-C6 heteroaryl ring) or fused ring (i.e. rings which share an adjacent pair of atoms) group having in the ring(s) one or more atoms, such as, for example, nitrogen, oxygen, and sulfur and, in addition, having a completely conjugated pi-electron system. In some embodiments, the terms “heteroaryl” and “C5-C6 heteroaryl” are used herein interchangeably. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazol, pyridine, pyrrole, oxazole, indole, purine, and the like.

As used herein, the terms “halo” and “halide”, which are referred to herein interchangeably, describe an atom of a halogen, that is fluorine, chlorine, bromine, or iodine, also referred to herein as fluoride, chloride, bromide, and iodide.

The term “haloalkyl” describes an alkyl group as defined above, further substituted by one or more halide(s).

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### EXAMPLES

### Example 1

### Conjugation of Phosphorylcholine to BOC-TYR

### 1) Preparation of Diazonium Salt

4-Aminophenyl (2-(trimethylammonio)ethyl) phosphate (50 mg, 0.18 mmol)) was dissolved in IM aqueous HCl (1 mL), cooled in an ice-water bath and sodium nitrite (12.6 mg, 0.18 mmol) was added in a single batch. The resulting solution was stirred at 0° C. for 30 min.

### 2) Azo Coupling

A new mixture was prepared with BOC-L-tyrosine (107 mg, 0.38 mmol) in NaHCO3(1M)+NaOH buffer (pH 10) (3.3 mL)+acetonitrile (1.2 mL). The mixture was cooled in an ice-water bath. The diazonium salt mixture was added drop-wise. A red solution was formed. Stirring of this was continued at 0° C. for 6 minutes. The reaction mixture was acidified with IN aqueous HCl to pH=˜3.

The obtained solution was lyophilized overnight, and subsequently purified (e.g. by preparative MPLC), to obtain the compound:

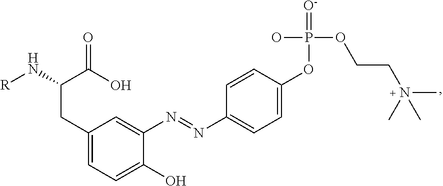

wherein R is Boc.

### Example 2

### Preparation of an Exemplary Compound of the Inveniton

### Preparation of Diazonium Salt:

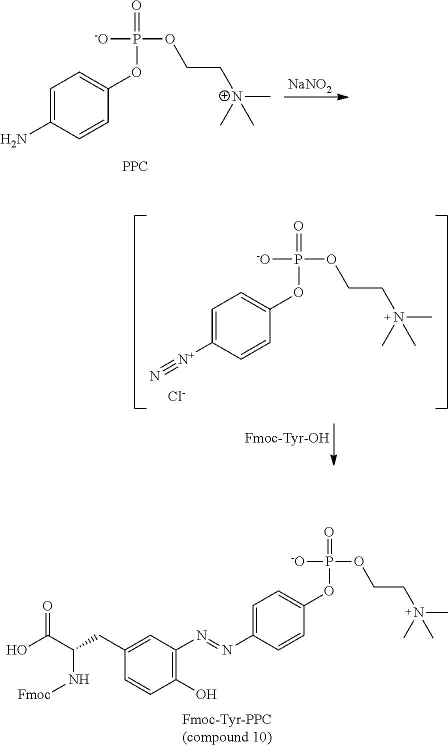

4-Aminophenyl (2-(trimethylammonio)ethyl) phosphate (250 mg, 0.912 mmol)) was dissolved in IM aqueous HCl (5 mL), cooled in an ice-water bath and sodium nitrite (62.9 mg, 0.912 mmol) was added in a single batch. The resulting solution was stirred at 0° C. for 30 min. Azo coupling, a new mixture was prepared with Fmoc-Tyr-OH (739 mg, 1.832 mmol) in saturated NaHCO3 (17 mL)+acetonitrile (12.5 mL). The resulting suspension/solution was cooled in an ice-water bath. The diazonium salt mixture was added drop-wise. Stirred at 0° C. The reaction mixture slowly turned yellow. After 5.5 h LCMS showed complete conversion. The reaction mixture was acidified with IN HCl to pH˜6, the yellowish suspension turned into a clear orange solution, which was lyophilized. This afforded 2.10 g. Dissolved in a mixture of DMSO/H20/MeCN (˜1:1:1) and purified in 5 runs by acidic preparative MPLC. The fractions were combined and lyophilized overnight, to obtain the desired product (compound 10).

### Example 3

### SPPS Synthesis of TRS

While facing difficulties with protection of the hydroxy group of compound 10, the inventors explored a novel strategy for SPPS synthesis of TRS:

The inventors initiated the SPPS synthesis by implementing the N-protected (Fmoc) phosphorylcholine modified tyrosine (e.g. compound 10) 200 mg of compound 10 were loaded onto the CTC resin. In brief, 2-Chlorotrityl chloride resin (1.0-1.2 mmol/g, 200-400 mesh) (450 mg, 1.441 mmol) was allowed to swell in dichloromethane (12 mL) by rocking for 30 min. The solvent was removed and a solution of (S,E)-4-((5-(2-((((9H-fluoren-9-yl)methoxy)carbonyl)amino)-2-carboxyethyl)-2-hydroxyphenyl)diazenyl)phenyl(2-(trimethylammonio)-ethyl) phosphate (200 mg, 0.290 mmol) in dichloromethane (12 mL) containing DIPEA (0.177 mL, 1.016 mmol) (substrate did not dissolve in DCM, after addition of DIPEA a solution was obtained) was added.

After 17 h the solvent was removed and the resin was washed with dichloromethane (3×10 mL, each washing step >2 minutes). The capping solution (CH2C12:MeOH:DIPEA 9:1:0.5) was added (10.5 mL) and the resin was rocked for 1 hour. Then the resin was washed with dichloromethane (3×10 mL) and dried in vacuo.

This resin was then split into equal portions in order to investigate a number of conditions for the subsequent chemistry in parallel, aimed at preventing the formation of the previously found tyrosine O-acylation, as witnessed by the isolation of compound 13 (see Scheme 2). The different reaction conditions were outlined in Table 1 (see below).

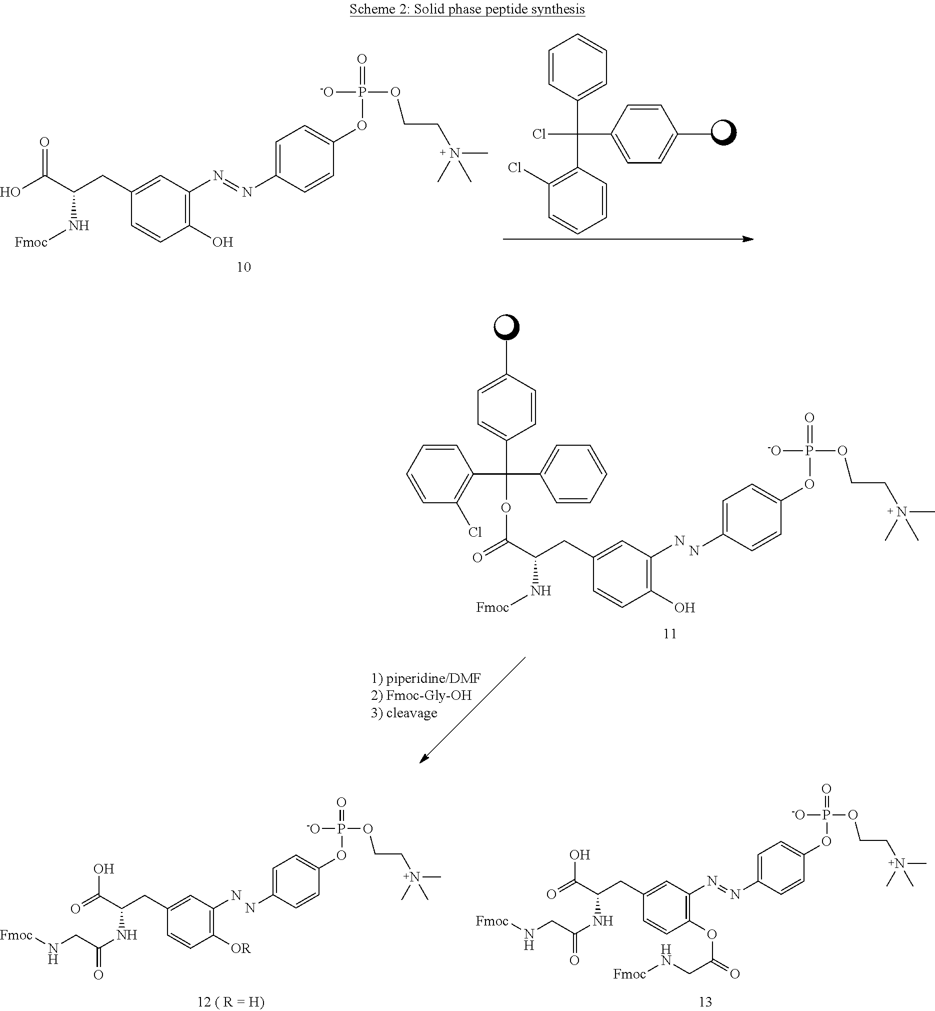

TABLE 1
exemplary coupling conditions tested

| Entry | Conditions |
|---|---|
| a | 4 eq. Fmoc-Gly-OH/HBTU/HOBt/DIPEA in DMF |
| b | 1.5 eq. Fmoc-Gly-OH/HBTU/HOBt/DIPEA in DMF |
| c | 4 eq. Fmoc-Gly-OH/DIC in DCM |
| d | 4 eq. Fmoc-Gly-OSu in DMF |

As shown in Table 1, various coupling conditions have been tested. Entries a-c resulted in the formation of a substantial amount of the byproduct (13). An improvement was obtained by using Fmoc-Gly-OSu in DMF (entry d). In this case the formation of byproduct (13) was reduced to only 3% relative to the desired compound 12. Nonetheless, neither of these methods was capable of suppressing the formation of 13 completely, therewith still posing a risk for further peptide synthesis, as this may lead to the accumulation of byproducts (compound 13).

Surprisingly, the inventors found that the byproduct (or phenolic ester byproduct, represented by compound 13 in Scheme 3) can be cleaved under standard Fmoc deprotection conditions with piperidine or with DBU in DMF, affording compound 15 cleanly, as illustrated below:

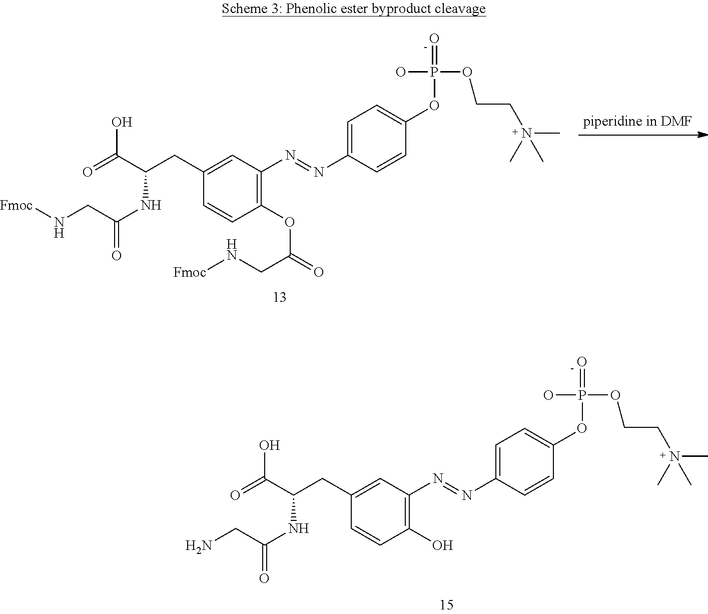

The inventors further successfully implemented additional amine bases (besides piperidine and DBU) for the cleavage of the by-product. Numerous amine bases have been successfully implemented for the cleavage of the by-product including primary and secondary amines, guanidine based compounds and amidine based compounds. Furthermore, the inventors observed that tertiary amines can be also implemented for the cleavage of the by-product, although tertiary amines sometimes require longer reaction time and/or subsequent repetition of the by-product cleavage steps. To this end, the inventors found that primary and secondary amines, guanidine based compounds and amidine based compounds have been characterized by improved reactivity and thus resulted in a more efficient by-product cleavage (e.g. resulting in an improved yield and/or purity of the compound of interest), as compared to tertiary amines under similar reaction conditions.

The amine bases which have been successfully tested by the inventors (at least about 95% by-product cleavage) are as follows: piperidine, DBU, cyclohexylamine, ethanolamine, pyrrolidine, morpholine, 4-methylpiperidine, tetramethylguanidine, and DBN. The amine bases have been implemented in a form of 5-20% solution in DMF (about 30 min incubation time). The inventors further observed that pyrrolidine, piperidine, DBU, cyclohexylamine, and ethanolamine have a superior efficiency (almost 100% yield) with respect to the cleavage of the by-product. Furthermore, it has been found that primary and secondary amines, guanidine based compounds and amidine based compounds (specifically, pyrrolidine, piperidine, DBU, cyclohexylamine, and ethanolamine) resulted in an almost complete Fmoc deprotection, so that the compound 15 has been obtained in 99-100% yield.

Upon loading of the compound 10 on the resin so as to obtain compound 11 (see Scheme 2) the peptide coupling with Fmoc-Gly-OH was repeated on solid phase under the standard conditions, after which an Fmoc deprotection was performed. Next, another peptide coupling step with Boc-Arg(Pbf)-OH was performed (Scheme 4, steps 1-4). Then, the resin was divided into 2 portions. The first portion was treated directly with HFIP for resin cleavage (step 5a), which afforded a mixture of compounds 16 and 17. The second batch was first treated with piperidine in DMF (step 5b), in order to cleave the phenolic ester. Then, the resin cleavage step was performed (step 6), which afforded the desired tripeptide 16 in very high purity, without a trace amount of di-coupled compound 17.

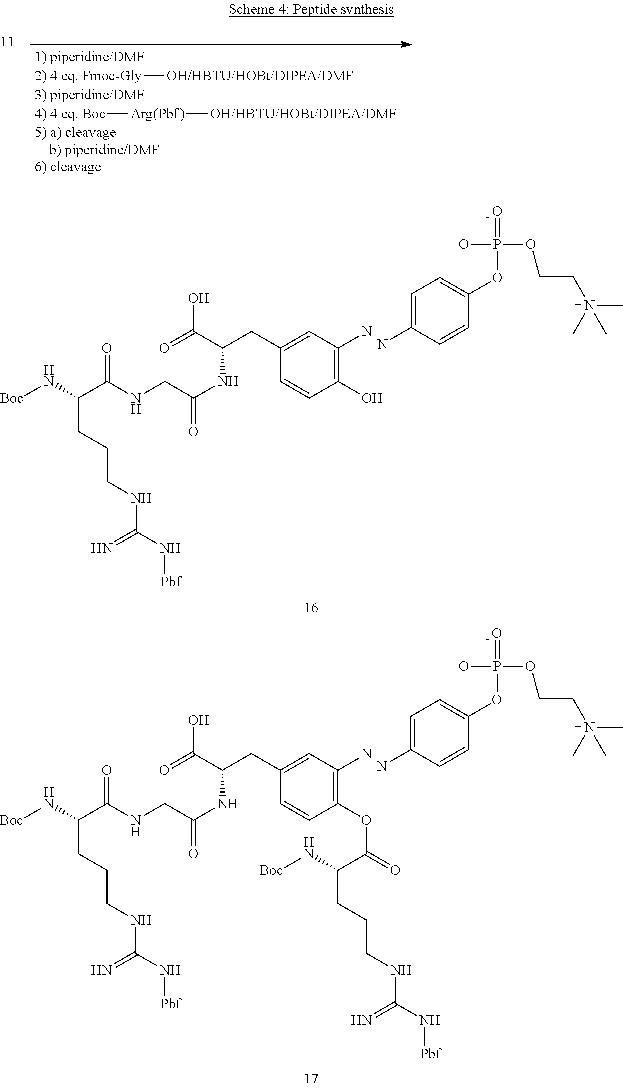

These result pave the way for solid phase synthesis of longer peptides under standard SPPS conditions, in which the problem of O-acylation on the free tyrosine OH is efficiently overcome by cleavage of the phenolic ester byproduct during the Fmoc deprotection step with piperidine in DMF.

A standard non-limiting procedure for SPPS synthesis of TRS is described hereinbelow.

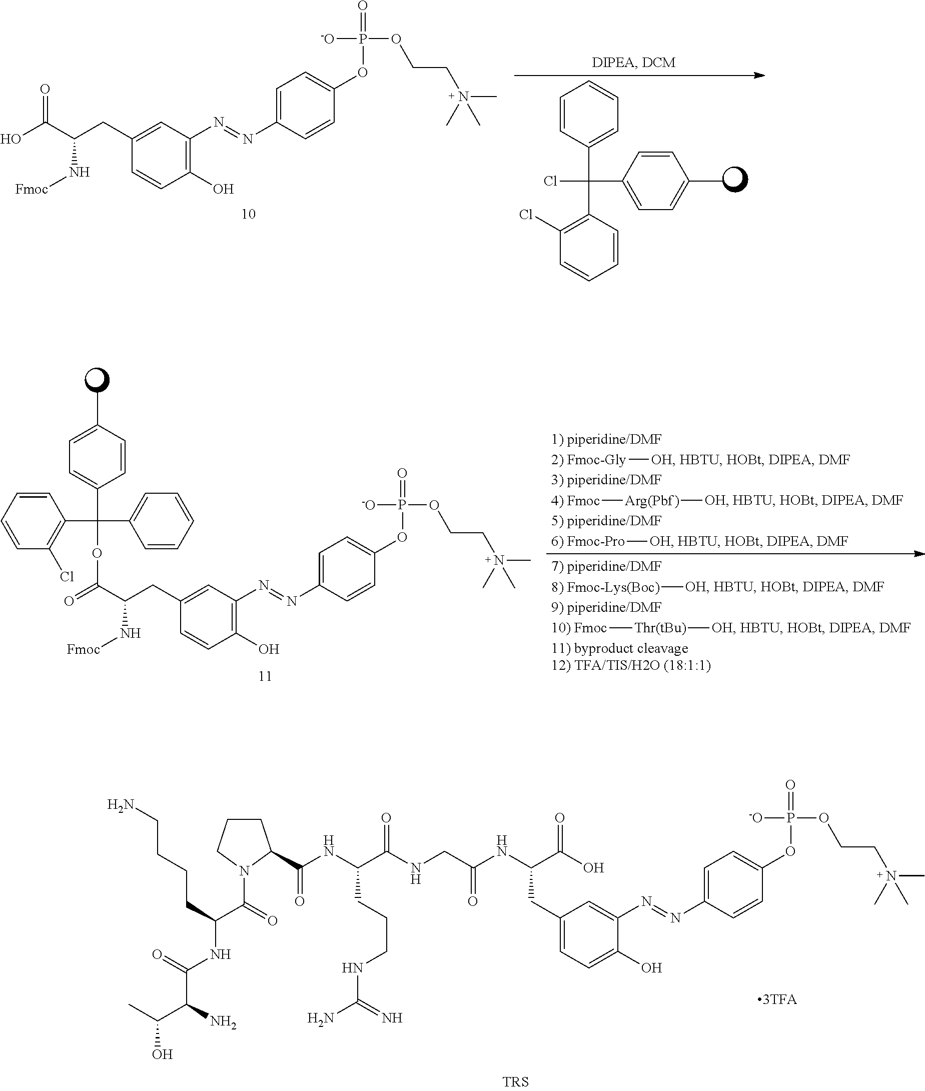

The compound 10 (Scheme 2) has been loaded on the solid support as described hercinabove, thereby resulting in the formation of compound 11.

Fmoc deprotection: the resin bound to the compound 10 (compound 11) was rocked in piperidine 20% (w/w) in peptide grade DMF (2 mL, 4.05 mmol) for 30 min. The piperidine solution was removed and the resin was washed with DMF (5 mL, peptide grade). This sequence was repeated for 2 times.

In the case of Boc-based SPPS synthesis, a Boc protected alternative of compound 10 can be used. The SPPS synthesis is almost identical to the Fmoc-based SPPS synthesis, however, the Boc cleavage is performed under acidic condition, usually comprising 50% TFA in DCM. Exact condition for Boc cleavage are well-known in the art. It is appreciated, that Boc-based SPPS requires a solid support compatible therewith. Solid supports for Boc- based SPPS are well-known in the art.

The resin-bound Fmoc-tyrosine-PPC (11) was then used for the solid phase peptide synthesis sequence towards TRS (Scheme 10). Each amino acid was coupled under standard conditions, using HBTU and HOBt with DIPEA in DMF. In some embodiments, each coupling step was performed for multiple times (e.g. twice).

Phenolic ester byproduct removal and cleavage of the peptide:

The resin was rocked in piperidine 20% (w/w) in peptide grade DMF (2 mL, 4.05 mmol) for 30 min, and then washed with DMF (2x3 mL). This sequence was repeated twice, resulting in an almost complete removal of the byproduct (as confirmed by analytical study). Alternatively, a DBU solution in DMF has been successfully implemented for the removal of the byproduct.

Finally, the peptide was cleaved from the resin using TFA/TIS/H2O (18:1:1), with simultaneous removal of all acid-labile protecting groups. The crude peptide was dissolved in water, after which Pbf residues could be easily removed by extraction with EtOAc and Et20. After lyophilization, crude TRS (TFA salt) was obtained in 63% yield with a purity of 88%. Purification by preparative HPLC afforded 240 mg (51% from 10) of the desired compound in high purity. Alternatively, the crude peptide (e.g. TRS and any one of the additional peptides disclosed hereinbelow) has been purified by preparative reversed phase MPLC to obtain the peptide of interest in high purity.

In some embodiments, byproduct removal and deprotection of the N-terminal protecting group are performed simultaneously (e.g. by applying the amine base cleavage solution to the peptide chain bound to the solid support, both the N-terminal protecting group and the byproduct are removed simultaneously).

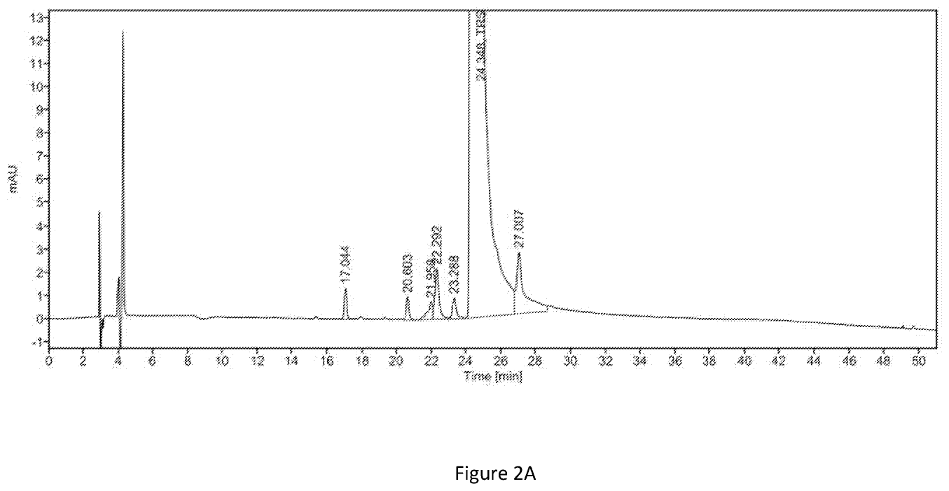

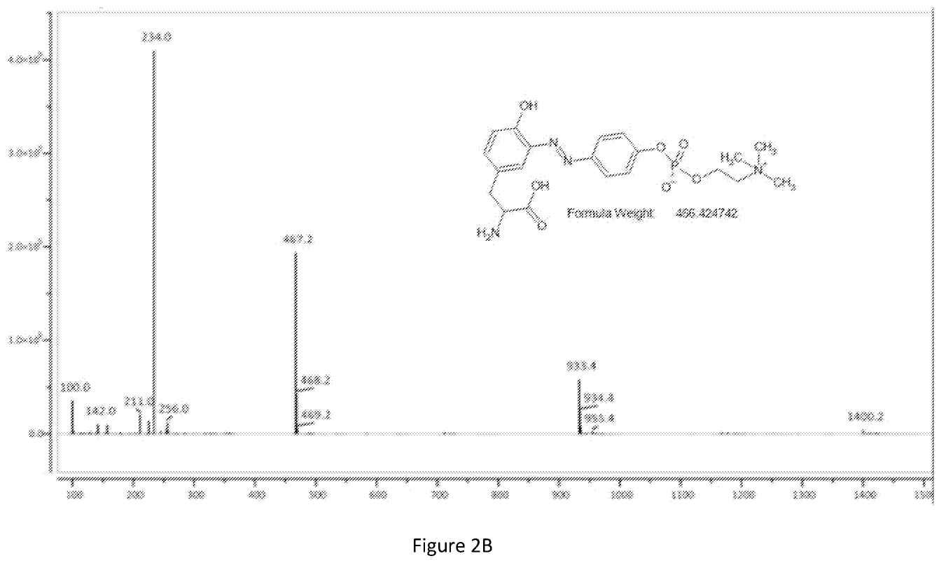

Additionally, the TRS synthesized as described herein has been further analyzed by HPLC and LC/MS to obtain an impurity profile thereof. The impurity profile has been further compared to the impurity profile of TRS synthesized by post-SPPS modification (diazotation) of Thr-Lys-Pro-Arg-Gly-Tyr, so as to couple the PC moiety to the phenol ring of tyrosine. Surprisingly, the inventors confirmed that the TRS synthesized as described herein has been characterized by a distinct impurity profile, as presented by FIGS. 1A and 2A. The inventors observed that whereas the TRS synthesized by post-SPPS modification has been characterized by 1.27 impurity, the TRS synthesized as described herein has been devoid of such impurity. Based on the proposed structure of the 1.27 impurity, it should be apparent that such impurity is specific to post-SPPS modification (diazotation).

Furthermore, the TRS synthesized as described herein has been characterized by a process specific impurity (0.7 impurity, disclosed hereinabove) corresponding to the compound of the invention devoid of protecting group(s). Accordingly, it is postulated that TRS (or any peptide bearing a diazotized tyrosine moiety) and synthesized as described herein, can be distinguished based on the process specific impurity(s), such as 0.7 impurity described hereinabove. Thus, the presence of an impurity comprising a diazotized tyrosine moiety

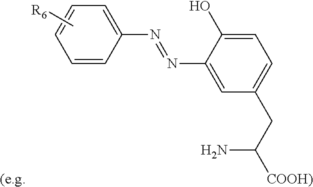

and/or a derivative thereof, is indicative of the peptide synthesized by the method of the invention.

To this end, the inventors successfully synthesized various peptides bearing tyrosine-PPC, by utilizing the compound of the invention (Fmoc-tyrosine-PPC) based on the method described herein. Exemplary peptides are as follows: H-Leu-Phe-Orn-Gly-TyrPPC-OH; H-Leu-Phe-TyrPPC-Orn-Gly-OH; and H-TyrPPC-Leu-Phe-Orn-Gly-OH. A skilled artisan will appreciate that any peptide sequence bearing a diazotized tyrosine moiety can be synthesized according to any one of the methods disclosed herein.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A compound or a salt thereof represented by Formula 1: 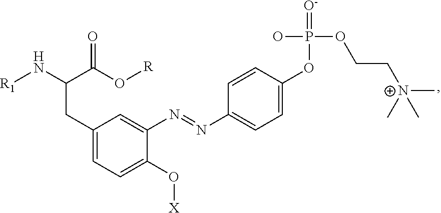
wherein: X is hydrogen, a phenol protecting group, or 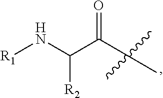
wherein R2 is a side chain of a natural or non-natural alpha amino acid protected or unprotected; each R1 is independently hydrogen or an amine protecting group; and
R is hydrogen, a carboxyl protecting group, a leaving group, a linker group of a solid phase, or is absent.

2. The compound of claim 1, wherein said amine protecting group comprises any one of 9-fluorenylmethyloxycarbonyl (Fmoc), Alloc, Dde, iv-Dde, benzyl, benzyloxycarbonyl, tert-butyloxycarbonyl (Boc), 2-[biphenylyl-(4)]-propyl-2-oxycarbonyl, dimethyl-3,5dimethoxybenzyloxycarbonyl, 2-(4-Nitrophenylsulfonyl)ethoxycarbonyl, 1,1-Dioxobenzo[b]thiophene-2-ylmethyloxycarbonyl, 2,7-Di-tert-butyl-Fmoc, 2-Fluoro-Fmoc, Nitrobenzenesulfonyl, Benzothiazole-2-sulfonyl, 2,2,2-Trichloroethyloxycarbonyl, Dithiasuccinoyl, p-Nitrobenzyloxycarbonyl.

3. The compound of claim 1, wherein said carboxyl protecting group comprises any one of tert-butyl ester, methyl ester, ethyl ester, benzyl esters, silyl esters 2-(Trimethylsilylethyl), (2-Phenyl-2-trimethylsiylyl)ethyl, 2-(Trimethylsilyl)isopropyl), allyl ester, 2-Chlorotrityl (2-Cl-Trt), 2,4-Dimethoxybenzyl, 2-Phenylisopropyl, 9-Fluorenylmethyl, Dmab, Carbamoylmethyl, Phenacyl, p-Nitrobenzyl, 4,5-Dimethoxy-2-nitrobenzyl, 1,1-Dimethylallyl.

4. The compound of claim 1, wherein said phenol protecting group comprises any one of triisopropylsilyl ether (TIPS), tert-Butyldimethylsilyl ether (TBDMS), methyl ether, Benzyl ether (Bn), methoxymethyl acetal (MOM), 2-(Trimethylsilyl)ethoxy]methyl acetal, tert-butyl ether, 2-chlorotrityl, trityl, benzyl, benzyloxycarbonyl, Boc.

5. The compound of claim 1, wherein said leaving group comprises any one of halo, hydroxy-succinimide, hydroxybenzotriazole, pentafluorophenol, imidazolecarbonate, O-acylisourea.

6. The compound of claim 1, wherein said compound is represented by Formula 2: 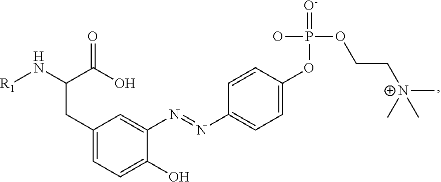
wherein R1 comprises said amine protecting group.

7. The compound of claim 6, wherein R1 is Fmoc or Boc.

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. A peptide having a following structure: 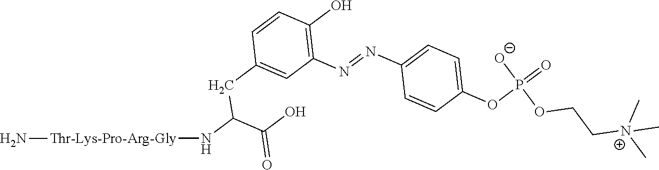
and being characterized by a chemical purity of at least 95% and wherein the peptide further comprises a trace amount of an impurity characterized by (i) MW of 466.4 Da; by (ii) a relative retention time (RRT) of about 0.7or both (i) and (ii).

23. The peptide of claim 22, wherein the impurity is 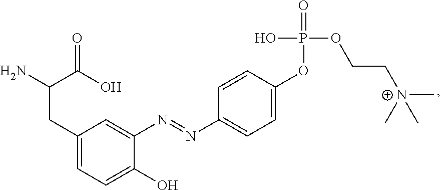 [SMILES: C[N+](C)(C)CCOP(=O)(O)Oc1ccc(/N=N/c2cc(CC(N)C(=O)O)ccc2O)cc1]
including any salt thereof.

24. The peptide of any one of claim 22, wherein the peptide is devoid of an impurity characterized by (i) by MW of 1263.5 Da, (ii) a relative retention time (RRT) of about 1.27; or both (i) and (ii).

25. The peptide of claim 22, wherein the RRT of the impurity is determined via an analytical Method A.

26. A method of synthesizing a peptide sequence of interest comprising diazotized tyrosine represented by Formula 3A: 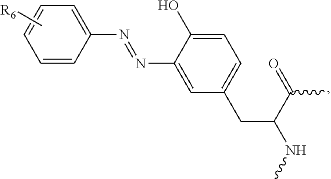
wherein R6 represents one or more substituent, and wherein at least one of the wavy bonds represents an attachment point to the peptide sequence of interest; and another wavy bond represents an attachment point to (i) any one of OH, O⁻, R1, N-protecting group, acyl, OR, and H, (ii) to the peptide sequence of interest; the method comprising:
(i) coupling a compound: 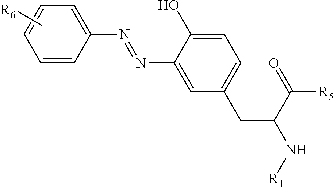
to a propagating peptide chain, wherein R1 comprises an amine protecting group, and R5 is OH, O;, hydrogen, an active ester, or a linker group attached to a solid phase and wherein (a) the propagating peptide chain, or (b) the compound is attached to the solid phase; and
(ii) contacting said solid phase with an amine base; thereby obtaining said peptide sequence of interest linked to the solid phase.

27. The method of claim 26, wherein said amine base is an Fmoc deprotecting agent.

28. The method of claim 26, wherein the amine base is selected from a primary amine, a secondary amine, a tertiary amine, a guanidine-based compound, and an amidine-based compound, including any combination thereof.

29. The method of claim 26, further comprising: (iii) deprotecting the amine protecting group to obtain a deprotected N-terminal amine; and (iv) coupling a subsequent N-protected amino acid to the deprotected N-terminal amine; wherein the steps (iii) and (iv) are performed prior to performing the step (ii).

30. The method of claim 29, wherein the steps (iii) and (iv) are performed subsequently and are optionally repeated one or more times.

31. The method of claim 26, further comprising performing a cleavage of the peptide sequence of interest linked to the solid phase, to obtain the peptide sequence of interest.

32. The method of claim 26, wherein R6 is or comprises 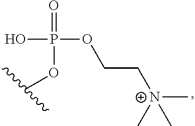 [SMILES: *OP(=O)(O)OCC[N+](C)(C)C]
and wherein the peptide sequence of interest is [chemical structure]
